(19) United States
(12) Patent Application Publication
(10) Pub. No.: US 2026/0041395 A1
(43) Pub. Date: Feb. 12, 2026

(54) METHOD AND APPARATUS FOR FOCUSED ULTRASOUND PRESSURE FIELD BASED ON MRI IMAGE, AND LEARNING METHOD FOR FOCUSED ULTRASOUND PRESSURE FIELD

(71) Applicant: UIF (University Industry Foundation), Yonsei University, Seoul (KR)

(72) Inventors: Kyungho YOON, Seoul (KR); Minjee SEO, Seoul (KR); Minwoo SHIN, Seoul (KR); Won-Yong SHIN, Seoul (KR)

(21) Appl. No.: 19/273,912

(22) Filed: Jul. 18, 2025

(30) Foreign Application Priority Data
Aug. 9, 2024 (KR) 10-2024-0106674

(51) Int. Cl. A61B 8/08; A61B 5/055; A61B 8/00

(52) U.S. Cl. CPC A61B 8/0875; A61B 5/055; A61B 8/4416

(57) ABSTRACT

A method for predicting a focused ultrasound pressure field based on an MRI image may include the steps of: obtaining input data including MRI image data of a skull shape, position data of a FUS transducer, and input free field sound pressure field data, in which the free field sound pressure field means a sound pressure field formed when ultrasound is propagated in a homogeneous medium; inputting the obtained MRI image data of a skull shape, position data of the FUS transducer, and the input free field sound pressure field data into a sound pressure field prediction module; and driving the sound pressure field prediction module to output in real-time the ultrasound pressure field data formed within the skull by the FUS applied by the FUS transducer.

### CROSS-REFERENCE TO RELATED APPLICATIONS

This application claims priority under 35 U.S.C. § 119(a) to Korean Patent Application No. 10-2024-0106674, filed on Aug. 9, 2024, with the Korean Intellectual Property Office, the disclosure of which is incorporated herein in its entirety by reference.

### BACKGROUND

### 1. Technical Field

The present disclosure relates to a method and apparatus for predicting a focused ultrasound (FUS) pressure field based on MRI images, and a learning method for predicting a FUS pressure field. More specifically, the present disclosure relates to a method and apparatus for predicting a FUS pressure field based on MRI images, which can predict in real-time the shape of an ultrasound pressure field formed within a cranial cavity according to a given shape of a skull and the position of a FUS transducer, and a learning method for predicting a FUS pressure field.

### 2. Description of the Related Art

FUS is used to treat a variety of areas as it can perform medical practices non-invasively by sonicating concentrated sound energy on a local area within living tissue. In order to perform non-invasive treatment using FUS, it is necessary to be able to generate ultrasound focus to a desired area. However, ultrasound is invisible and undergoes complex interactions such as reflection, refraction, and scattering when propagating through heterogeneous biological tissues, due to variations in acoustic impedance between different tissue types.

To solve these problems, a magnetic resonance-guided FUS (MRgFUS) system has been developed, which visualizes ultrasound focus by detecting temperature changes through magnetic resonance thermometry module, but the equipment has the problem of high cost. In addition, in certain fields (e.g., brain stimulation, etc.), low-intensity FUS (LIFU) is used, but because the temperature change is small, the use of the MRgFUS system can be problematic.

In addition, there is a system that displays the focal position of a FUS transducer on a medical image obtained in advance using real-time optical tracking equipment through image-guided FUS (neuro-navigation), but it has the limitation of not considering the effect of changes in the position and intensity of the focal point due to the skull.

### SUMMARY OF THE INVENTION

An object of the present disclosure is to provide a method and apparatus for predicting a FUS pressure field based on MRI images, which can predict the shape of an ultrasound pressure field formed within a skull according to a given shape of a skull and the position of a FUS transducer in real-time, and a learning method for predicting a FUS pressure field.

Another object of the present disclosure is to provide a method and apparatus for predicting a FUS pressure field based on MRI images, which predict the shape of an ultrasound pressure field formed within a skull in real-time by using MRI image data of a skull shape, position data of a FUS transducer, and input free field sound pressure field data as inputs to a sound pressure field prediction module, and a learning method for predicting a focused ultrasound pressure field.

In order to achieve the above objects, according to one embodiment of the present disclosure, a method for predicting a FUS pressure field based on an MRI image is disclosed, comprising the steps of: obtaining input data including MRI image data of a skull shape, position data of a FUS transducer, and input free field sound pressure field data, wherein the free field sound pressure field means a sound pressure field formed when ultrasound is propagated in a homogeneous medium; inputting the obtained MRI image data of a skull shape, position data of the FUS transducer, and the input free field sound pressure field data into a sound pressure field prediction module; and driving the sound pressure field prediction module to output in real-time the ultrasound pressure field data formed within the skull by the FUS applied by the FUS transducer.

In order to achieve the above objects, according to one embodiment of the present disclosure, an apparatus for predicting a focused ultrasound pressure field based on an MRI image is disclosed, comprising: an input data acquisition unit that obtains input data including MRI image data of a skull shape, position data of a FUS transducer, and input free field sound pressure field data, wherein the free field sound pressure field means a sound pressure field formed when ultrasound is propagated in a homogeneous medium; a memory in which the sound pressure field prediction module is stored; an input unit that receives the obtained input data as an input of the sound pressure field prediction module; an output unit that outputs the ultrasound pressure field data formed within the skull by the FUS applied by the FUS transducer in real-time based on the obtained input data; and a control unit that generally controls the input data acquisition unit, the memory, the input unit, and the output unit, and executes the sound pressure field prediction module to derive the ultrasound pressure field data formed within the skull according to the input data as output data.

In order to achieve the above objects, according to one embodiment of the present disclosure, a learning method for predicting a focused ultrasound pressure field is disclosed, comprising the steps of: preprocessing CT image data of a skull shape and MRI image data of a skull shape; inputting input data including the preprocessed image data, position data of a FUS transducer, and input free field sound pressure field data as inputs of a sound pressure field prediction module; calculating ultrasound pressure field data formed within the skull by FUS applied by a FUS transducer based on the CT image data; and outputting the ultrasound pressure field data as an output of the sound pressure field prediction module.

According to one embodiment of the present disclosure, the shape of an ultrasound pressure field formed within the skull can be predicted in real-time based on MRI image data of the skull shape and the position of a FUS transducer without a CT image.

According to one embodiment of the present disclosure, in addition to MRI image data and position data of a FUS transducer, free field sound pressure field data is further utilized, so that the shape of an ultrasound pressure field formed within the skull can be more accurately derived.

### BRIEF DESCRIPTION OF THE DRAWINGS

FIG. 1 is a block diagram showing an apparatus for predicting a focused ultrasound pressure field based on an MRI image related to an embodiment of the present disclosure.

FIG. 2 is a flowchart showing a learning method for predicting a focused ultrasound pressure field based on an MRI image related to an embodiment of the present disclosure.

FIG. 3 is a flowchart showing step S210 of FIG. 2.

FIG. 4 is a diagram for explaining a preprocessing process of CT image data and MRI image data in step S310 of FIG. 3.

FIG. 5 is a diagram for explaining position setting of a FUS transducer in step S320 of FIG. 3.

FIG. 6 is a diagram for explaining a process of obtaining an ultrasound pressure field within the skull in step S330 of FIG. 3.

FIG. 7 is a diagram for explaining a process of obtaining free field sound pressure field data in step S330 of FIG. 3.

FIG. 8 is a flowchart showing a method for predicting a focused ultrasound pressure field based on an MRI image related to an embodiment of the present disclosure.

### DETAILED DESCRIPTION OF THE INVENTION

Hereinafter, a method and apparatus for predicting a focused ultrasound pressure field based on an MRI image, and a learning method for predicting a focused ultrasound pressure field, related to an embodiment of the present disclosure, will be described with reference to the drawings.

The singular expressions “a”, “an”, and “the” used in the present specification include plural referents unless the context clearly indicates otherwise. In the present application, terms such as “consisting of” or “comprising” should not be construed as necessarily including all of the various elements or various steps described in the specification, and it should be construed that some of the components or steps may not be included or additional components or steps may be further included.

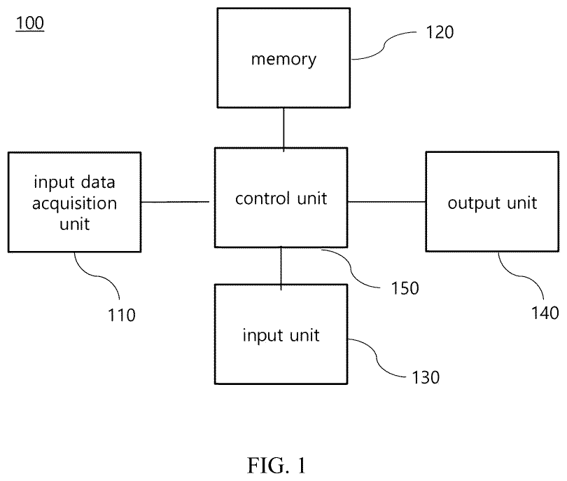

FIG. 1 is a block diagram showing an apparatus for predicting a focused ultrasound pressure field based on an MRI image related to an embodiment of the present disclosure.

As illustrated, the apparatus 100 for predicting a sound pressure field may include an input data acquisition unit 110, a memory 120, an input unit 130, an output unit 140, and a control unit 150.

The input data acquisition unit 110 may obtain input data used as input for a intracranial FUS pressure field prediction module (not shown, hereinafter referred to as a ‘sound pressure field prediction module’). The input data may be received from an external device or generated internally. The input data may include MRI image data of a skull shape, position data of a FUS transducer, and input free field sound pressure field data.

The input data of the sound pressure field prediction module may have different types or numbers of data in the learning stage and the inference stage.

The memory 120 may store data. The memory 120 may store a sound pressure field prediction module. In this case, the sound pressure field prediction module may be implemented based on artificial intelligence. Specifically, the sound pressure field prediction module may be implemented with artificial intelligence based on a convolutional neural network (CNN) or a Swin Transformer. In addition, the memory 120 may store input data input to the sound pressure field prediction module and output data output through the sound pressure field prediction module.

The input unit 130 may receive input data obtained from the input data acquisition unit 110 as input to the sound pressure field prediction module.

The output unit 140 may output the result value of the sound pressure field prediction module corresponding to the input data in a form that can be confirmed by the user.

The control unit 150 may include a processor. The control unit 150 may control the input data acquisition unit 110, the memory 120, the input unit 130, and the output unit 140 as a whole. The control unit 150 may control the execution of the sound pressure field prediction module stored in the memory 120 so that result data is derived according to data input through the input unit 130 and the output unit 140, and the derived result data is output through the output unit 140.

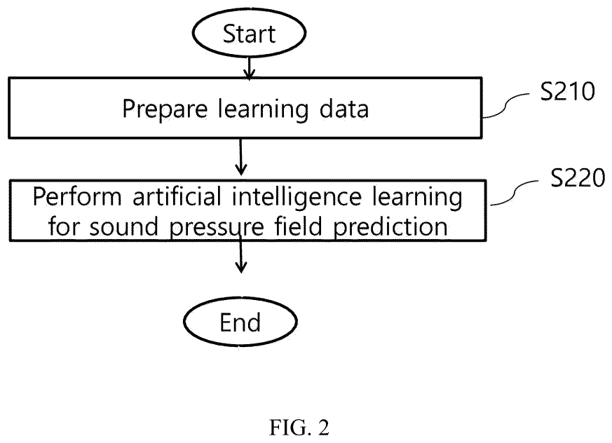

FIG. 2 is a flowchart showing a learning method for predicting a focused ultrasound pressure field based on an MRI image related to an embodiment of the present disclosure.

Learning data for learning the artificial intelligence of the sound pressure field prediction module may be prepared (S2100). The learning data includes skull shape data, position data of a FUS transducer, sound pressure field (acoustic pressure field) shape data within the skull, and free field sound pressure field data.

The skull shape data is data regarding the three-dimensional shape of the skull. The skull shape data may be provided in the form of a three-dimensional image of the skull. The skull shape data may be an image captured through CT (computed tomography) or an image captured through MRI.

The position data of the FUS transducer is data about the point where the ultrasound transducer is positioned with respect to the skull. The position data of the ultrasound transducer may include coordinate data indicating three-dimensional coordinates at which the ultrasound transducer is positioned with respect to the skull, and angle data indicating the angle at which the FUS transducer is positioned with respect to the skull (i.e., the angle at which ultrasound is irradiated with respect to the skull).

FUS may irradiate concentrated acoustic energy in a local area within biological tissue. Accordingly, FUS is used for therapeutic purposes by applying acoustic energy through specific regions of the human body. In particular, FUS may facilitate non-invasive neuromodulation when applied to the brain, and may thus be utilized in therapeutic brain stimulation and in the treatment of neurological disorders.

FUS can be divided into high-intensity FUS (HIFU) and low-intensity FUS (LIFU) depending on its intensity. HIFU irreversibly changes the condition of the target area to achieve therapeutic effects, such as thrombolysis, extracorporeal shockwave, thermal ablation, and boiling histotripsy.

On the other hand, LIFU is used in applications such as drug delivery through opening the blood-brain barrier and non-invasive brain stimulation. In addition, LIFU has recently demonstrated potential for the treatment of neurological diseases such as epilepsy, Alzheimer's, and Parkinson's disease.

For FUS treatment to be effective, ultrasound must be delivered precisely to the desired area. However, ultrasound is invisible and undergoes complex interactions such as reflection, refraction, and scattering when propagating through heterogeneous biological tissues. In particular, due to the wave nature of ultrasound, distortion occurs at boundaries where regions of different acoustic properties meet. Consequently, severe aberration occurs when ultrasound propagates through the skull or porous structures, making transcranial therapeutic applications of FUS particularly challenging.

On the other hand, the transcranial focused ultrasound pressure field prediction apparatus 100 according to one embodiment of the present disclosure reflects the aberration as the ultrasound propagates and passes through an area including the skull, and provides ultrasound pressure field data formed on the inside of the cranial cavity when the ultrasound transducer is positioned on the outside of the skull corresponding to the input skull shape data. Accordingly, a user performing a medical procedure can effectively perform a medical procedure using ultrasound through the ultrasound pressure field data provided by the transcranial focused ultrasound pressure field prediction apparatus.

Intracranial sound pressure field shape data is data on the shape of the ultrasound propagated from the ultrasound transducer traveling across the skull, i.e., the area where the brain is located. Intracranial sound pressure field shape data may be provided in the form of a three-dimensional image or a two-dimensional image with respect to a specific reference plane.

A free field sound pressure field refers to a sound pressure field formed when an ultrasound is propagated in a homogeneous medium (e.g., water). For example, the free field sound pressure field data is ultrasound pressure field data when the entire simulation area is assumed to be water during a simulation process using numerical analysis. For the free field sound pressure field, a reference free field sound pressure field is first calculated, and then the reference free field sound pressure field is rotated to generate free field sound pressure field data to be used as an input value (hereinafter referred to as “input free field sound pressure field data”). This will be described later in more detail.

Therefore, the intracranial sound pressure field shape data may be prepared in the form of a learning data set to be paired with the skull shape data, the input free field sound pressure field data, and the position data of the ultrasound transducer that applies ultrasound to the skull.

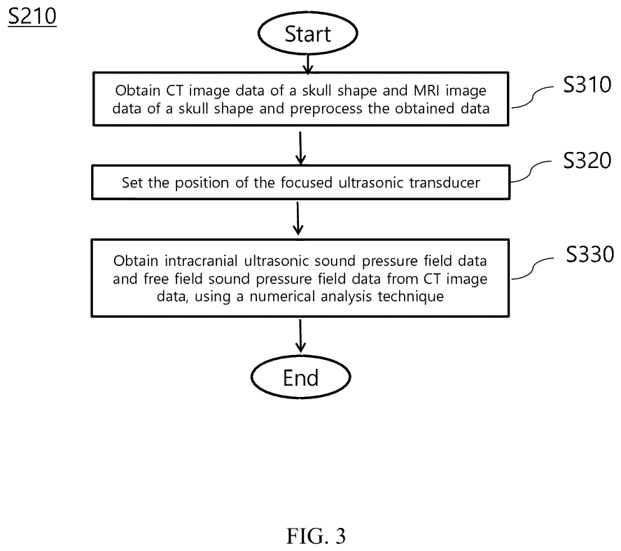

FIG. 3 is a flowchart showing step S210 of FIG. 2.

First, CT image data of a skull shape and MRI image data of a skull shape may be obtained, and the obtained data may be preprocessed (S310).

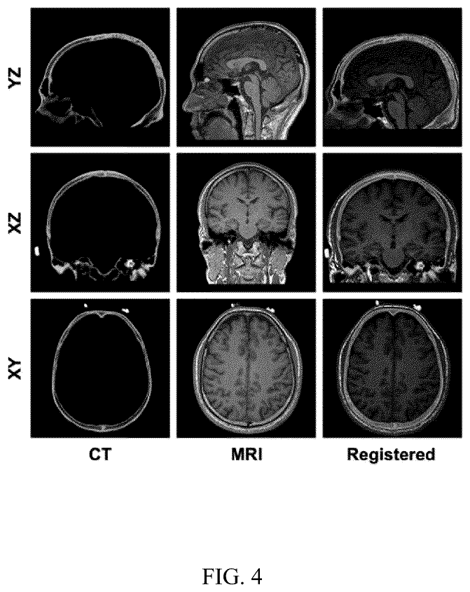

FIG. 4 is a diagram for explaining a preprocessing process of CT image data and MRI image data in step S310 of FIG. 3.

For example, CT image data of skull shapes and MRI image data of skull shapes for multiple patients may be obtained.

CT images may have a specific spatial resolution per voxel unit (e.g., spatial resolution of 0.5 mm×0.5 mm×0.5 mm).

The area below the frontal bone may be removed to process the image to have a specific spatial resolution (e.g., 225 mm×225 mm×150 mm (450×450×300 voxel grid)).

In FIG. 4, the MRI image has a resolution of 1 mm×1 mm×1 mm per voxel grid, and was resampled using the Lanczos interpolation method to have a resolution of 0.5 mm×0.5 mm×0.5 mm per voxel unit to align the spatial resolution with the CT image.

To align the spatial coordinates between the CT and MRI images, the CT and MRI images were registered using the normalized mutual information method.

The position of the focused ultrasonic transducer may be set (S320).

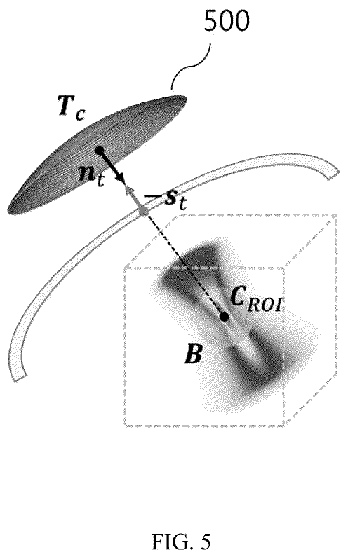

FIG. 5 is a diagram for explaining position setting of a FUS transducer in step S320 of FIG. 3.

In order to set the position of a FUS transducer 500, the position and direction vector of a FUS transducer satisfying predetermined conditions may be obtained.

For example, the focal distance from the target point (C_{ROI}) may be set to a specific distance (e.g., 83 mm). The position of the FUS transducer 500 may be set such that the angle between the skull surface normal vector and the direction vector of the transducer at the point where the ultrasound beam meets the skull is less than or equal to 10 degrees.

A specific method for obtaining the position and direction vector of the transducer 500 is as follows.

Extract the coordinates of an area with a radius of a certain size (e.g., 45 mm) from a predefined thalamus location and define this as a set B.

Extract a set S of skull surface area coordinates, and triangulate the extracted area to find the normal vector −Sₜ in the triangulated area.

For all elements of S and B, find the direction vector nₜ between them.

Find a point C_{ROI} in B such that the angle between Sₜ and nₜ is less than a predetermined angle (e.g., 10°), and find a coordinate (T_{C}) at a point that is a predetermined distance (e.g., 83 mm) away from the C_{ROI} in the −nₜ direction. Then, the center position of the transducer 500 may be defined as T_{C}, and the direction vector may be defined as nₜ.

Then, using a numerical analysis technique, intracranial ultrasound pressure field data and free field sound pressure field data may be obtained from CT image data (S330).

In order to simulate the intracranial sound pressure field using numerical analysis techniques, the shape of the patient's skull (skull model) through which the ultrasound will be transmitted must be obtained.

To obtain the skull model, the patient's skull 3D CT image data may be classified according to the HU value (φ) to extract the cortical bone, trabecular bone, and the intracranial region as 3D voxel coordinates.

A simulation space may be constructed by assigning acoustic properties (sound speed (c), density (ρ), and attenuation coefficient (a) of the medium) appropriate for each tissue to each voxel.

The acoustic properties of a tissue according to HU values_{i, j, k} at voxel grid coordinates i, j, k can be defined by the following Equation 1.

$$[ Equation 1 ]$$

$${c}_{i , j , k} = \{ 1500 m / s , for {ϕ}_{i , j , k} \leq 0 , 2140 m / s , for 0 < {ϕ}_{i , j , k} < 1000 ,$$

$$for 1000 < \text{?}$$

$${ρ}_{i , j , k} = \{ 1000 kg / \text{?} , for {ϕ}_{i , j , k} \leq 0 , 1000 + 1.19 {ϕ}_{i , j , k} kg / \text{?} ,$$

$$for \text{?} < {ϕ}_{i , j , k} < 1000 , 2190 kg / \text{?} ,$$

$$\text{?} = 33 Np / m , for 0 < \text{?}$$

$$\text{?} \text{indicates text missing or illegible when filed}$$

c_{i,j,k} is the speed of ultrasound, and ρ_{i,j,k} is the density of ultrasound. Here, φ_{i,j,k}<=0 is water, 0<φ_{i,j,k}<=1000 is trabecular bone, and φ_{i,j,k}>1000 is cortical bone. a_{i,j,k} is an attenuation coefficient.

To simplify the simulation, it is assumed that the acoustic properties of the intracranial region are the same as those of water. In addition, it is assumed that the point to be targeted by the FUS transducer 500 is the thalamus region of the brain, and by registering the CT and MRI images, the location of the thalamus region may be extracted as three-dimensional voxel coordinates from the MRI image.

The position of the transducer 500 may include simulation coordinate data representing the three-dimensional coordinates of the center position (T_{C}) and simulation angle data representing the angle at which the transducer 500 is positioned with respect to a skull model (not shown).

The simulation angle data can be defined as a normal vector of the exit surface. The simulation coordinate data and the simulation angle data may correspond to coordinate data and angle data of the ultrasonic transducer 500, respectively.

The region of interest (C_{ROI}) is a region for obtaining shape data of the propagation of ultrasound irradiated from the transducer 500 and may be set to have a preset volume. As an example, the region of interest (C_{ROI}) may be set to a size of 56 mm×56 mm×56 mm.

To perform ultrasound propagation modeling within a region of interest (C_{ROI}), the Westervelt-Lightill equation, as expressed in Equation 2 below, may be used as the governing equation.

$$\text{?} p - \frac{\text{?}}{\text{?}} \frac{\text{?}}{\text{?}} - \frac{\text{?}}{\text{?}} \frac{\text{?}}{\text{?}} = 0 with \text{?} = 2 a \sqrt{\frac{\text{?}}{\text{?}} + \text{?}} \qquad [ Equation 2 ]$$

$$\text{?} \text{indicates text missing or illegible when filed}$$

p is the sound pressure, c is the speed of sound in the medium, t is time, a is the attenuation coefficient of the medium, and f is the frequency of the sound wave.

To numerically approximate the solution of the governing equation, the finite-difference time-domain (FDTD) method is used. Discretizing the governing equation according to the FDTD method, the following Equation 3 can be obtained.

$$\text{?} = {P}_{i , j , k}^{n} - {C}_{i , j , k}^{p} Δ ( \text{?} + \text{?} + \text{?} - \text{?} , \qquad [ Equation 3 ]$$

$$\text{?} \text{indicates text missing or illegible when filed}$$

wherein,

$${P}_{i , j , k}^{n}$$

is the sound pressure at voxel grid coordinate i,j,k at time unit n,

$$\text{?}$$

$$\text{?} \text{indicates text missing or illegible when filed}$$

is the at voxel grid coordinate i,j,k,

$$\text{?} , \text{?} , \text{?}$$

$$\text{?} \text{indicates text missing or illegible when filed}$$

are the wave speeds in x, y, z directions at voxel grid coordinate i,j,k at time unit n+½, and A_{i,j,k} is the attenuation coefficient.

The sound speed, wave speed, and attenuation coefficient can be calculated using the following Equation 4, respectively.

$$Δ {V}_{i , j , k}^{x , n + \frac{1}{2}} = {V}_{i , j , k}^{x , n + \frac{1}{2}} - \text{?} , \qquad [ Equation 4 ]$$

$$Δ {V}_{i , j , k}^{y , n + \frac{1}{2}} = {V}_{i , j , k}^{y , n + \frac{1}{2}} - {V}_{i , j - 1 , k}^{y , n + \frac{1}{2}} ,$$

$$Δ {V}_{i , j , k}^{x , n + \frac{1}{2}} = \text{?} - {V}_{i , j , k - 1}^{z , n + \frac{1}{2}} ,$$

$${V}_{i , j , k}^{x , n + \frac{1}{2}} = \text{?} - \text{?} ( {P}_{i + 1 , k}^{n} - {P}_{i , j , k}^{n} ) ,$$

$${V}_{i , j , k}^{y , n + \frac{1}{2}} = {V}_{i , j , k}^{y , n + \frac{1}{2}} - \text{?} ( \text{?} - \text{?} ) ,$$

$${V}_{i , j , k}^{z , n + \frac{1}{2}} = \text{?} - \text{?} ( \text{?} - \text{?} ) ,$$

$$\text{?} = 2 Δ t \text{?} \sqrt{\frac{\text{?}}{4 {π}^{2} {f}^{2}} + \text{?}} ,$$

$$\text{?} = {ρ}_{i , j , k} {c}_{i , j , k}^{2} \frac{Δ t}{Δ x} ,$$

$$\text{?} = \frac{2 Δ t}{( \text{?} + \text{?} ) Δ x} ,$$

$$\text{?} = \frac{2 Δ t}{( \text{?} + \text{?} ) Δ x} ,$$

$$\text{?} = \frac{2 Δ t}{( \text{?} + \text{?} ) Δ x}$$

$$\text{?} \text{indicates text missing or illegible when filed}$$

wherein, c_{i,j,k}, ρ_{i,j,k}, α_{i,j,k} are the sound speed, density, and attenuation coefficient of the medium at voxel grid coordinates i, j, k, respectively, x is the grid size in the spatial domain, and t is the grid size in the time domain.

For the stability of the numerical solution, t is defined to satisfy the CFL condition (Equation 5) below.

$$Δ t \leq \frac{Δ x}{\sqrt{3} c} \qquad [ Equation 5 ]$$

wherein, Δt is the time interval, Δx is the discretized space interval, and c is the wave speed.

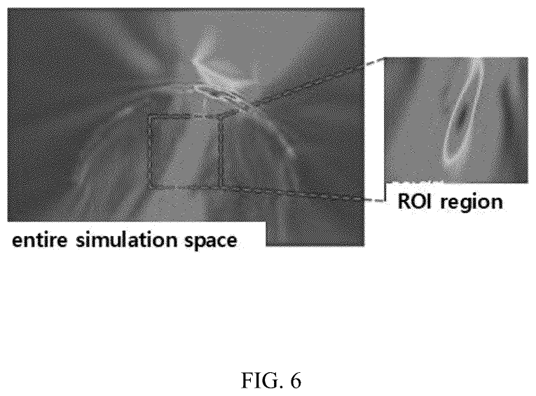

FIG. 6 is a diagram for explaining a process of obtaining an ultrasound pressure field within the skull in step S330 of FIG. 3.

Referring to FIG. 6, the intracranial sound pressure field for the transducer 500 position in the entire simulation area can be calculated using Equations 4 and 5.

In addition, based on the region of interest (C_{ROI}), a region of interest (ROI) can be defined with spatial dimensions of a specific size (e.g., 112×112×112 voxel size).

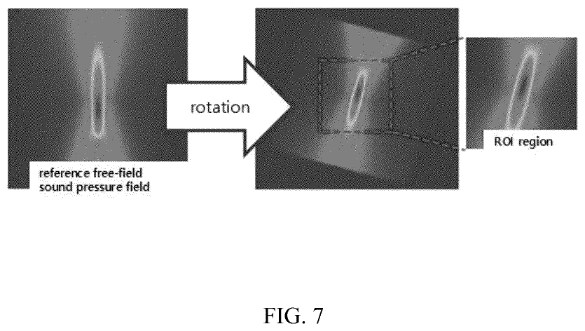

FIG. 7 is a diagram for explaining a process of obtaining free field sound pressure field data in step S330 of FIG. 3. The free field sound pressure field may refer to a sound pressure field formed when ultrasound propagates in a homogeneous medium (e.g., water).

FIG. 7 shows an example of the process of obtaining free field sound pressure field data. For example, to calculate the free field sound pressure field, the angle of the transducer is defined as [0,0,1], and the acoustic characteristics of the entire simulation area are defined as water to first calculate the reference sound pressure field (reference free-field sound pressure field).

Afterwards, when calculating the intracranial sound pressure field, by rotating the reference free-field sound pressure field according to the angle of the transducer 500 defined, an input free-field sound pressure field data set that is paired with the intracranial sound pressure field can be obtained. The region of interest (ROI) can also be defined for the free-field sound pressure field in the same way as in FIG. 6.

Since the input free-field sound pressure field can be obtained by rotating the reference free-field sound pressure field according to the angle of the transducer 500, the input free-field sound pressure field data can be obtained more simply.

Referring back to FIG. 2, as described above, when the learning data set is prepared, artificial intelligence learning for sound pressure field prediction can be performed (S220).

The input data input into the artificial intelligence are free-field sound pressure field data, MRI patch, and transducer position data.

The MRI patch may be obtained by defining a region of interest (ROI) having the same spatial dimensions as the sound pressure field centered on the skull surface coordinates for each location of the transducer 500 in an MRI image registered with the CT image described in FIG. 3.

The ground-truth data is the intracranial sound pressure field calculated through simulations of Equations 4 and 5.

Once learning is complete, a focused ultrasound pressure field can be output in real time according to input data through the apparatus for predicting a focused ultrasound pressure field based on an MRI image.

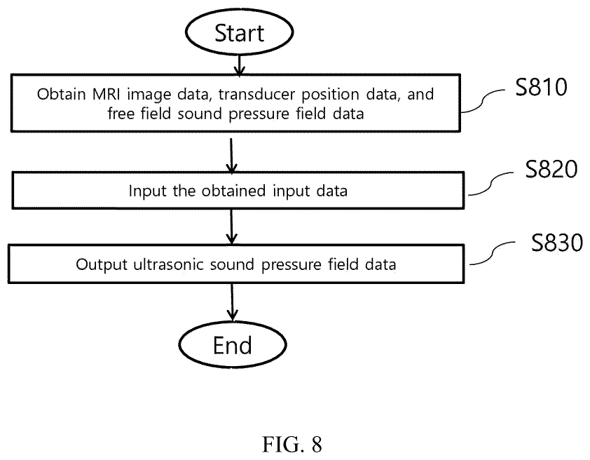

FIG. 8 is a flowchart showing a method for predicting a focused ultrasound pressure field based on an MRI image related to an embodiment of the present disclosure.

The input data acquisition unit 110 may obtain input data of the sound pressure field prediction module stored in the memory 120 (S10). The input data may include MRI image data of a skull shape, position data of a FUS transducer, and input free field sound pressure field data. In particular, by using the input free field sound pressure field data as input data, the sound pressure field prediction module can perform operations in real-time.

The input unit 130 may receive the input data and input it into the sound pressure field prediction module (S820).

The output unit 140 may output intracranial sound pressure field data corresponding to the input data as an output value (S830).

As described above, according to one embodiment of the present disclosure, it is possible to predict the shape of an ultrasound pressure field formed within the skull in real-time based on MRI image data of the skull shape and the position of a FUS transducer without a CT image.

According to one embodiment of the present disclosure, in addition to MRI image data and position data of a FUS transducer, free field sound pressure field data is further utilized, so that the shape of an ultrasound pressure field formed within the skull can be more accurately derived.

The method and apparatus for predicting a focused ultrasound pressure field based on MRI images, and the learning method for predicting a focused ultrasound pressure field, described above, are not limited to the configurations and methods of the embodiments described above, and all or some of the aforementioned embodiments may selectively be configured in combination so that various modifications may be made in the aforementioned embodiments.

What is claimed is:

1. A method for predicting a focused ultrasound pressure field based on an MRI image, comprising the steps of:
obtaining input data including MRI image data of a skull shape, position data of a focused ultrasound (FUS) transducer, and input free field sound pressure field data, in which free field sound pressure field means a sound pressure field formed when ultrasound is propagated in a homogeneous medium;
inputting the obtained MRI image data of the skull shape, the position data of the FUS transducer, and the input free field sound pressure field data into a sound pressure field prediction module; and
driving the sound pressure field prediction module to output in real time ultrasound pressure field data formed within a skull by a focused ultrasound applied by the FUS transducer.

2. The method according to claim 1,
wherein the step of obtaining input data includes the steps of:
calculating a reference free-field sound pressure field based on a reference position of the FUS transducer; and
deriving the input free field sound pressure field data by rotating the reference free-field sound pressure field by a predetermined angle.

3. The method according to claim 2,
wherein the predetermined angle for rotating the reference free-field sound pressure field is determined based on current position data of the FUS transducer.

4. The method according to claim 2,
wherein the position data of the FUS transducer includes coordinate data indicating a three-dimensional coordinate at which the FUS transducer is positioned with respect to the skull and angle data indicating an angle at which the FUS transducer is positioned with respect to the skull.

5. The method according to claim 3,
wherein a transcranial focused ultrasound pressure field prediction module is provided by artificial intelligence based on a convolutional neural network (CNN) or a Swin Transformer.

6. The method according to claim 5,
wherein the artificial intelligence uses learning data including the MRI image data of the skull shape, CT image data of the skull shape, the position data of the FUS transducer, the input free field sound pressure field data, and the ultrasound pressure field data formed within the skull, and
the MRI image data, the position data of the FUS transducer, and the input free field sound pressure field data are used as inputs, and the ultrasound pressure field data corresponding thereto is used as output,
so that learning is performed to predict a shape of the focused ultrasound pressure field according to the MRI image data of the skull shape and the position data of the FUS transducer.

7. An apparatus for predicting a focused ultrasound pressure field based on an MRI image, comprising:
an input data acquisition unit that obtains input data including MRI image data of a skull shape, position data of a focused ultrasound (FUS) transducer, and input free field sound pressure field data, in which free field sound pressure field means a sound pressure field formed when ultrasound is propagated in a homogeneous medium;
a memory in which a sound pressure field prediction module is stored;
an input unit that receives the obtained input data as an input of the sound pressure field prediction module;
an output unit that outputs in real time ultrasound pressure field data formed within a skull by the FUS applied by the FUS transducer based on the obtained input data; and
a control unit that generally controls the input data acquisition unit, the memory, the input unit, and the output unit, and executes the sound pressure field prediction module to derive the ultrasound pressure field data formed within the skull according to the input data as output data.

8. The apparatus according to claim 7,
wherein the input data acquisition unit
calculates a reference free-field sound pressure field based on a reference position of the FUS transducer, and
derives the input free field sound pressure field data by rotating the reference free-field sound pressure field by a predetermined angle.

9. The apparatus according to claim 8,
wherein the predetermined angle for rotating the reference free-field sound pressure field is determined based on current position data of the FUS transducer.

10. The apparatus according to claim 7,
wherein the position data of the FUS transducer includes coordinate data indicating a three-dimensional coordinate at which the FUS transducer is positioned with respect to the skull and angle data indicating an angle at which the FUS transducer is positioned with respect to the skull.

11. The apparatus according to claim 9,
wherein a transcranial focused ultrasound pressure field prediction module is provided by artificial intelligence based on a convolutional neural network (CNN) or a Swin Transformer.

12. The apparatus according to claim 11,
wherein the artificial intelligence uses learning data including the MRI image data of the skull shape, CT image data of the skull shape, the position data of the FUS transducer, the input free field sound pressure field data, and the ultrasound pressure field data formed within the skull, and
the MRI image data, the position data of the FUS transducer, and the input free field sound pressure field data are used as inputs, and the ultrasound pressure field data corresponding thereto is used as output,
so that learning is performed to predict a shape of the focused ultrasound pressure field according to the MRI image data of the skull shape and the position data of the FUS transducer.

13. A learning method for predicting a focused ultrasound pressure field, comprising the steps of:
preprocessing CT image data of a skull shape and MRI image data of a skull shape;
inputting input data including preprocessed image data, position data of a focused ultrasound (FUS) transducer, and input free field sound pressure field data as inputs of a sound pressure field prediction module;
calculating ultrasound pressure field data formed within a skull by FUS applied by the FUS transducer based on the CT image data; and
outputting the ultrasound pressure field data as an output of the sound pressure field prediction module.

14. The learning method according to claim 13,
wherein the step of preprocessing includes a step of registering the CT image data of the skull shape and the MRI image data of the skull shape.
